(19) United States
(12) Patent Application Publication
(10) Pub. No.: US 2020/0155343 A1
(43) Pub. Date: May 21, 2020

(54) BODY WARMING DEVICE

(71) Applicant: Mohsen Zamanigohar, Burnaby (CA)

(72) Inventor: Mohsen Zamanigohar, Burnaby (CA)

(21) Appl. No.: 16/691,556

(22) Filed: Nov. 21, 2019

Related U.S. Application Data
(60) Provisional application No. 62/917,115, filed on Nov. 21, 2018.

(51) Int. Cl. A61F 7/00

(52) U.S. Cl. CPC A61F 7/0085; A61F 2007/0061

(57) ABSTRACT

The present invention relates to a self-contained body warming device, comprising: a mask, the mask dimensioned to cover a user's mouth and nose, and comprising one or more attachments to secure the mask over the user's mouth and nose; and a flexible hose having a first end and a second end, the hose coupled at the first end to the mask, and the hose dimensioned such that the second end extends to the user's torso when the mask is secured; wherein as the user exhales during breathing, exhaled air travels along the flexible hose from the first end to the second end, thereby warming the user's body at the location of the second end.

### CROSS-REFERENCE TO RELATED APPLICATIONS

This nonprovisional application claims priority based upon the following prior United States Provisional Patent Application entitled: Natural Heater, Application No. 62/917,115 filed Nov. 21, 2018, in the name of Mohsen Zamanigohar and, which is hereby incorporated by reference for all purposes.

### FIELD OF THE INVENTION

The present specification relates generally to body warming, and more particularly to a self-contained body warming device.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

Keeping oneself at a comfortable temperature when outdoors is a continual challenge for many people, whether working outdoors, spectating at an event, or even just walking or waiting for transportation. Commonly, people will rub or blow on their hands to provide temporary warmth using their own body heat in the expelled air.

However, such measures generally prove ineffective for the rest of the body. Maintain a comfortable temperature for the body, whether intermittent or ongoing, when existing clothing proves insufficient, is not addressed. While various electrically powered heating devices are known in the art, they generally are considered cumbersome and may present a risk to the user via overheating.

Therefore, it would be desirable to have a body warming device which may provide the desired comfort by leveraging natural internal body heat. It would also be desirable for such a device to minimal interfere with the natural breathing process.

Accordingly, there remains a need for improvements in the art.

### SUMMARY OF THE INVENTION

In accordance with an aspect of the invention, there is provided a self-contained body warming device.

According to an embodiment of the invention, there is provided a self-contained body warming device, comprising: a mask, the mask dimensioned to cover a user's mouth and nose, and comprising one or more attachments to secure the mask over the user's mouth and nose; and a flexible hose having a first end and a second end, the hose coupled at the first end to the mask, and the hose dimensioned such that the second end extends to the user's torso when the mask is secured; wherein as the user exhales during breathing, exhaled air travels along the flexible hose from the first end to the second end, thereby warming the user's body at the location of the second end.

According to a further embodiment of the invention, the mask may further comprise one or more one-way valves such that the user inhales air through the one-way valves. Additionally, when the hose is coupled to the mask via a coupling, the coupling may include a one-way valve, the one-way valve permitting the passage of exhaled air into the hose.

According to a still further embodiment of the invention, the mask may be secured to a piece of headwear, such as a helmet or ski hat, instead of around the user's head.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a front view of a body warming device view according to an embodiment;

FIG. 2 is a side view of the device of FIG. 1 worn on a human body; and

FIG. 3 is an exploded perspective view of a coupling and hose according to an embodiment.

Like reference numerals indicated like or corresponding elements in the drawings.

### DETAILED DESCRIPTION OF THE EMBODIMENTS

The present invention relates generally to body warming, and more particularly, without limitation, to a self-contained body warming device.

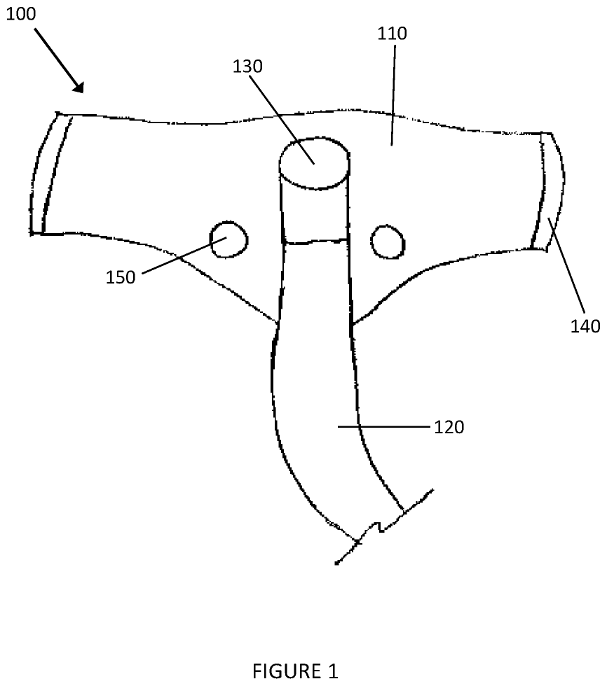

According to an embodiment as shown in FIG. 1, a body warming device 100 comprises a mask 110 coupled to a first end of a flexible hose 120. The mask 110 and hose 120 may be coupled via a separate coupling 130. The mask may include one or more attachment mechanisms 140 to secure the mask 110 to the use's head and over the user's mouth and nose. The attachment mechanisms may include a strap (fixed or detachable) and various types of fasteners, including, but not limited to, snap fasteners, magnets and hook-and-loop fasteners.

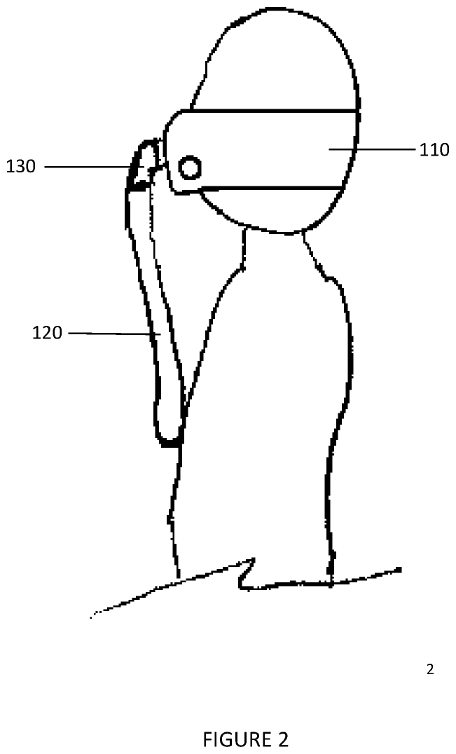

In operation, as shown in FIG. 2, the mask 110 covers the user's mouth and nose, and a second end of the flexible hose 120 extends over the user's torso. As the user exhales, the exhaled air is dissipated over the user's torso, thereby at least temporarily warming the user. The mask and hose are dimensioned such that the natural breathing process is minimally disrupted, allowing the use to warm their body through natural breathing and exertion.

Additionally, one or more one-way valves 150 may be provided in the mask to draw in exterior air when the user inhales, while forcing the exhaled air into flexible hose 120. This also ensure the user is breathing properly fresh and oxygenated air and not rebreathing exhaled air. Similarly, a separate one-way valve working in the opposite direction may be incorporated into coupling 130 to prevent the user from rebreathing exhaled air.

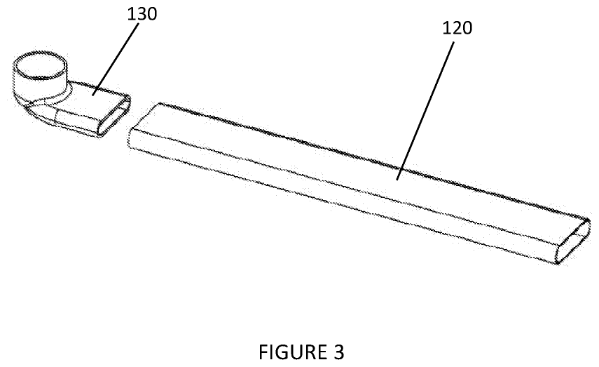

Referring to FIG. 3, coupling 130 and flexible hose 120 may be separately removable and detachable from mask 110 and each other. This permits replacement for cleaning or if either component is damaged. Additionally, the device 100 may be supplied as a kit with multiple hoses 120 of varying lengths to accommodate different sized users and hose placements over the torso e.g. higher/lower and front/back. Alternatively, flexible hose 120 may be over an accordion-type or similar that permits adjustment of the length of hose 120.

While device 100 is described herein as independently attached to the user's head, alternatively the mask 110 may be secured to a piece of headwear, such as a helmet or ski hat, instead of to or around the user's head.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A self-contained body warming device, comprising:
a mask, the mask dimensioned to cover a user's mouth and nose, and comprising one or more attachments to secure the mask over the user's mouth and nose; and
a flexible hose having a first end and a second end, the hose coupled at the first end to the mask, and the hose dimensioned such that the second end extends to the user's torso when the mask is secured;
wherein as the user exhales during breathing, exhaled air travels along the flexible hose from the first end to the second end, thereby warming the user's body at the location of the second end.

2. The body warming device of claim 1, wherein the mask further comprises one or more one-way valves such that the user inhales air through the one-way valves.

3. The body warming device of claim 1, wherein the hose is coupled to the mask via a coupling, the coupling including a one-way valve, the one-way valve permitting the passage of exhaled air into the hose.

4. The body warming device of claim 1, wherein the one or more attachments comprise a detachable strap.

5. The body warming device of claim 4, wherein the detachable strap is secured via one or more of: magnets, hook-and-loop fasteners, and snap fasteners.

6. The body warming device of claim 1, wherein the hose is detachably coupled to the mask.

7. The body warming device of claim 3, wherein the coupling is detachably coupled to the mask.

8. The body warming device of claim 3, wherein the coupling is detachably coupled to the hose.

9. The body warming device of claim 1, wherein the mask is attachable to a piece of headwear.

10. The body warming device of claim 9, wherein the piece of headwear is a helmet.

11. The body warming device of claim 9, wherein the piece of headwear is a ski hat.

12. The body warming device of claim 1, wherein the hose is adjustable in length.
